(19) United States
(12) Patent Application Publication
(10) Pub. No.: US 2021/0191528 A1
(43) Pub. Date: Jun. 24, 2021

(54) Light-Emitting Device Applicable to Keyboard of Notebook Computer and Use Method Therefor

(71) Applicant: Pentagon Maple Leaf Information Technologies of Kunshan Co., Ltd., Kunshan (CN)

(72) Inventor: Lianhui Zhou, Zhangjiagang (CN)

(73) Assignee: Pentagon Maple Leaf Information Technologies of Kunshan Co., Ltd., Kunshan (CN)

(21) Appl. No.: 16/954,013

(22) PCT Filed: Sep. 18, 2018
(86) PCT No.: PCT/CN2018/106084

§ 371 Date: Jun. 15, 2020

(30) Foreign Application Priority Data
Sep. 10, 2018 (CN) 201811051709.9

(51) Int. Cl. G06F 3/02; G06F 3/023; H01H 13/83

(52) U.S. Cl. CPC G06F 3/0202; A61L 2/00; H01H 13/83; G06F 3/0231

(57) ABSTRACT

Light-emitting device applicable to keyboard of notebook computer and use method thereof, the device comprising a power adapter (1), a control box (3), a keyboard membrane (6) and a display screen (13); the keyboard membrane (6) is provided thereon with a key membrane (7), the key membrane (7) and the keyboard membrane (6) being integrally formed; the display screen (13) is disposed on the key membrane (7), and the display screen (13) is embedded on the key membrane (7); which is internally provided with a key slot (12), the key slot (12) and the key membrane (7) being integrally formed; the keyboard membrane (6) comprises an upper anti-static layer (26), a center base layer (27) and a heat conduction layer (28). The degree of intelligence of the device is high, the structure is well-designed, operation is simple, use is convenient and functions are comprehensive such that operation of a notebook computer is more convenient and faster, thereby improving the usage experience of the notebook computer, while antimicrobial and antipollution effects may be effective, the waterproof and anti-static capabilities of the notebook computer are improved, and practicability is strong.

### TECHNICAL FIELD

The present invention relates to the field of computers, and in particular to a light-emitting device applicable to a notebook computer keyboard and a method of using the same.

### ARTS OF BACKGROUND

Computer common name computer It is a modern electronic computer for high-speed computing. It can perform numerical calculations, logic calculations, and memory storage. Is able to follow the procedure Modern intelligent electronic equipment that processes large amounts of data automatically and at high speed. With the development of technology, computers can be seen everywhere in people's lives. A notebook computer is a portable computer. The light-emitting device of a notebook computer keyboard is one of the matching devices on a notebook computer, and is mainly used to improve the experience of the notebook computer. There are many such devices available on the market, but these devices are relatively low in intelligence, unreasonable in structural design, inconvenient to use and operate, and have a single function, and can not effectively help the operation of the notebook computer, affecting Practicality.

### SUMMARY OF THE INVENTION

The object of the present invention is to provide a light-emitting device applicable to a notebook computer keyboard and a method for using the same in order to solve the above problems. The invention has high intelligence, reasonable structure, convenient use and strong practicability.

The present invention achieves the above objects by the following technical solutions:

The invention relates to a light-emitting device applicable to a notebook computer keyboard, comprising a power adapter, a control box, a keyboard membrane and a display screen, wherein the keyboard membrane is provided with a button membrane, and the button membrane and the keyboard membrane are integrally formed The display screen is provided with the display screen, and the display screen is embedded on the button membrane. The button membrane is provided with a button slot, and the button slot and the button membrane are integrally formed. The keyboard membrane includes an upper antistatic layer, a central base layer and a heat conductive layer, the upper antistatic layer is disposed above the central base layer, the heat conductive layer is disposed under the central base layer, and the upper antistatic layer An upper waterproof layer is disposed above, an antibacterial layer is disposed above the upper waterproof layer, an anti-staining layer is disposed above the antibacterial layer, a lower antistatic layer is disposed under the heat conduction layer, and a lower antistatic layer is disposed under the lower antistatic layer a lower waterproof layer, a nano-adhesive layer disposed under the lower waterproof layer, the anti-stain layer, the anti-bacterial layer, the upper waterproof layer, the upper anti-static layer, the central base layer, the heat-conducting layer, The lower antistatic layer, the lower waterproof And bonding to the nano-adhesive layer by glue, the other side of the keyboard membrane is provided with a second connecting braiding wire, the second connecting braiding wire is embedded on the keyboard membrane, and the second connecting braiding wire The control box is provided with a power switch, a power switch is disposed on the control box, a snap connection is connected between the power switch and the control box, and a circuit board is disposed in the control box, and the circuit board passes a screw is fixed in the control box, the power switch and the circuit board are connected by wires, and the display screen is connected to the circuit board through the second connection braiding wire, and the other side of the control box a first connection flat wire is disposed, and the other end of the first connection flat wire is provided with a USB connector, and the USB connector is provided with a connection indicator light and an operation status indicator, the connection indicator light and the working status indicator Separably embedded in the USB connector, the USB connector, the connection indicator and the working status indicator are connected to the circuit board through the first connection flat wire, and the power supply is extended behind the control box Line, the power extension cord is another The power adapter is provided, and the model of the power adapter is LYD2400500, and the power adapter and the circuit board are connected by the power extension cable, and the power adapter is provided with a power plug, and the power plug is set A rubber spacer is disposed between the power extension cord and the power adapter on the power adapter, and the rubber spacer has an interference fit with the power extension cord and the power adapter, respectively.

Further, the circuit board is provided with a filter, the type of the filter is AHD34, and the filter is soldered on the circuit board.

In this way, the filter is used to prevent signal interference, and the filter and the circuit board are electrically connected by soldering.

Further, the filter is provided with a Wi-Fi chip on one side thereof, and the model of the Wi-Fi chip is ZBT-WM8302RT, and the Wi-Fi chip is soldered on the circuit board by soldering.

In this way, the Wi-Fi chip is used to receive and transmit a wireless signal, and the Wi-Fi chip and the circuit board are electrically connected by soldering.

Further, the other side of the filter is provided with a signal amplifier, the type of the signal amplifier is HDV-R45, and the signal amplifier is soldered on the circuit board by soldering.

In this way, the signal amplifier is used to amplify the enhanced signal, and the signal amplifier and the circuit board are electrically connected by soldering.

Further, a controller is disposed in front of the Wi-Fi chip, and the model of the controller is SPC-STW-26A1, and the controller is soldered on the circuit board by soldering.

In this way, the controller is used for control, and the controller and the circuit board are electrically connected by soldering.

Further, a processor is disposed in front of the filter, and the model of the processor is MT6995.

So set up, the processor is used to process signals and data.

Further, the processor is soldered to the circuit board by soldering.

In this way, the processor and the circuit board are electrically connected by soldering.

Further, a memory is disposed in front of the signal amplifier, and the model of the memory is TVS-882ST2.

So set up, the memory is used to store data.

Further, the memory is soldered to the circuit board by soldering.

In this way, the memory and the circuit board are electrically connected by soldering.

The invention also provides a method for using a light-emitting device applicable to a notebook computer keyboard, which is applied to the light-emitting device applicable to a notebook computer keyboard, and the specific use method comprises the following steps:

a. The keyboard membrane is pasted on the notebook computer keyboard through the nano-adhesive layer, the power adapter is connected to the power source through the power plug, and the power switch is turned on to connect the USB connector to the USB of the notebook computer. The interface is connected. After the connection is completed, the connection indicator is red. After the driver is installed, the working status indicator is green, and the connection indicator is off;

b. After the computer is ready, the parameters and functions of the device can be set by the driver software through the driver software. The device related data can be stored in the memory, so that the parameters and functions set are still available after the notebook computer is replaced, and the setting is avoided;

c. During use, the corresponding display screen on the button membrane can display corresponding shortcut function icons according to different shortcut keys according to different software, so that the software can be operated more conveniently, and the effect is more effective. At night, the display screen can illuminate and display its corresponding button name so that the notebook computer can be operated in a dark environment. When listening to music, the display screen can emit colorful lights according to the rhythm of the music;

d. The Wi-Fi chip can receive and transmit a wireless network signal, the signal amplifier can amplify the wireless signal, the filter can prevent signal interference, and the wireless connection between the mobile phone and the device can be established through setting, and the mobile phone is established with the mobile phone. After the wireless network is connected, the notebook computer can be turned on and off and other corresponding operations through the mobile phone, so that the notebook computer can be operated more conveniently, and the notebook user experience is improved;

e. Finally, the anti-contamination layer and the anti-bacterial layer can effectively resist anti-staining and anti-bacterial, and the upper anti-static layer and the lower anti-static layer can prevent electrostatic damage to electronic components, the upper waterproof layer and the The waterproof layer can ensure that the keyboard is waterproof, and the heat conduction layer can conduct heat, thereby assisting the notebook computer to dissipate heat.

Compared with the prior art, the beneficial effects of the present invention are as follows:

1. High degree of intelligence, reasonable structure, simple operation and convenient use;

2, full-featured, making the operation of the notebook computer more convenient and faster, thereby improving the experience of the notebook computer;

3, can be effective antibacterial and anti-contamination, and improve the waterproof and anti-static ability of the notebook computer, more practical.

### DRAWINGS

In order to more clearly illustrate the embodiments of the present invention or the technical solutions in the prior art, the drawings used in the embodiments or the description of the prior art will be briefly described below. Obviously, the drawings in the following description are only It is a certain embodiment of the present invention, and other drawings can be obtained from those skilled in the art without any inventive labor.

FIG. 1 is a schematic top plan view of a light-emitting device applicable to a notebook computer keyboard according to the present invention;

FIG. 2 is a bottom perspective view of a light-emitting device applicable to a notebook computer keyboard according to the present invention;

FIG. 3 is a partial enlarged view of a light-emitting device applicable to a notebook computer keyboard according to the present invention;

FIG. 4 is a perspective structural view of a USB connector of a light-emitting device applicable to a notebook computer keyboard according to the present invention;

FIG. 5 is a block diagram showing the internal structure of a control box of a light-emitting device applicable to a notebook computer keyboard according to the present invention;

FIG. 6 is a cross-sectional view of a keyboard membrane of a light-emitting device applicable to a notebook computer keyboard according to the present invention;

FIG. 7 is a flow chart showing the circuit structure of a light-emitting device applicable to a notebook computer keyboard according to the present invention.

The reference numerals are as follows:

| | |
|---|---|
| 1 | power adapter |
| 2 | power extension cable |
| 3 | control box |
| 4 | first connection flat wire |
| 5 | USB connector |
| 6 | keyboard membrane |
| 7 | button membrane |
| 8 | second connection braided wire |
| 9 | switch |
| 10 | power plug |
| 11 | rubber spacer |
| 12 | button slot |
| 13 | display |
| 14 | connection indicator |
| 15 | working status indicator |
| 16 | controller |
| 17 | processor |
| 19 | signal amplifier |
| 20 | filter |
| 21 | Wi-Fi chip |
| 22 | circuit board |
| 23 | anti-contamination layer |
| 24 | antibacterial layer |
| 25 | upper waterproof layer |
| 26 | upper anti-static layer |
| 27 | central base layer |
| 28 | thermal conductive layer |
| 29 | lower antistatic layer |
| 30 | lower waterproof layer |
| 31 | nano-adhesive layer. |

### EMBODIMENTS

In the description of the present invention, it is to be understood that the terms “center”, “longitudinal”, “transverse”, “upper”, “lower”, “front”, “back”, “left”, “right”, the orientation or positional relationship of the indications of “upright”, “horizontal”, “top”, “bottom”, “inside”, “outside”, etc. is based on the orientation or positional relationship shown in the drawings, only for the convenience of describing the present invention and the simplified description, which is not intended to indicate or imply that the device or elements in question have a definite orientation, configuration and operation in a particular orientation, which is not limiting the scope of the invention; moreover, the terms of “first”, “second” and the like mean descriptiveness only, and should not be construed as indicating or implying a relative importance or the number of technical characteristics. Thus, the definition of “first”, “second”, etc. may include one or more technical characteristics by explicitly or implicitly; in the description of the present invention, “plurality” means two or more unless otherwise stated, and “installation” and “connected” are understood in broad meanings, being fixed or detachable, mechanical or electrical, directly or indirectly connected through an intermediate medium, for example, unless otherwise explicitly defined. The specific meaning of the above terms in the present invention can be understood by a person with ordinary skill in the art.

The present invention will be further described below in conjunction with the accompanying drawings:

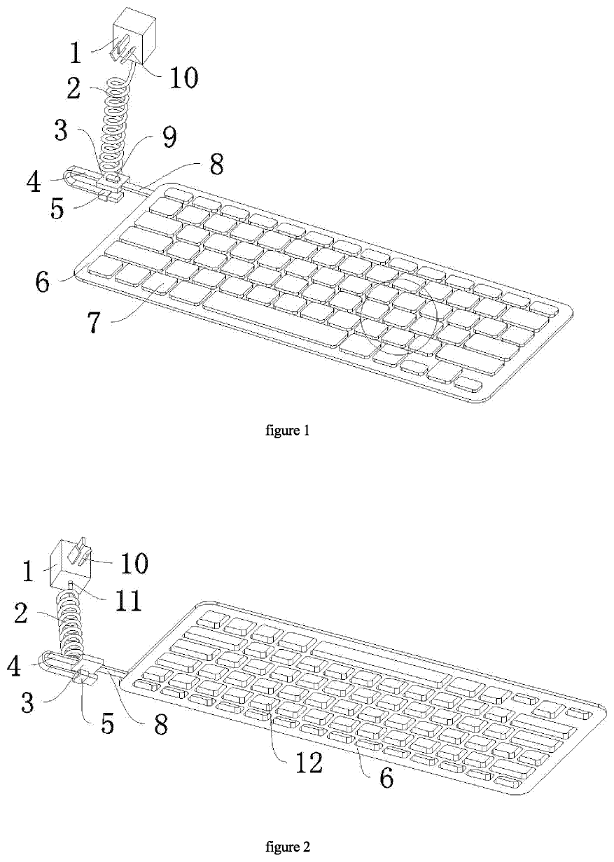

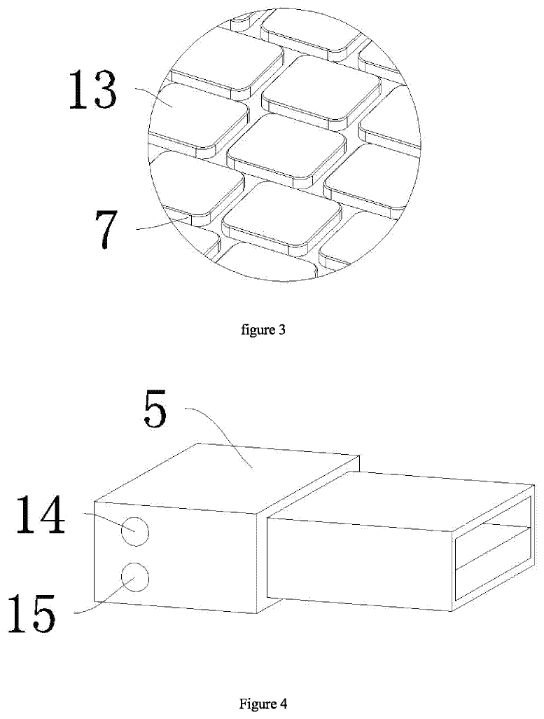

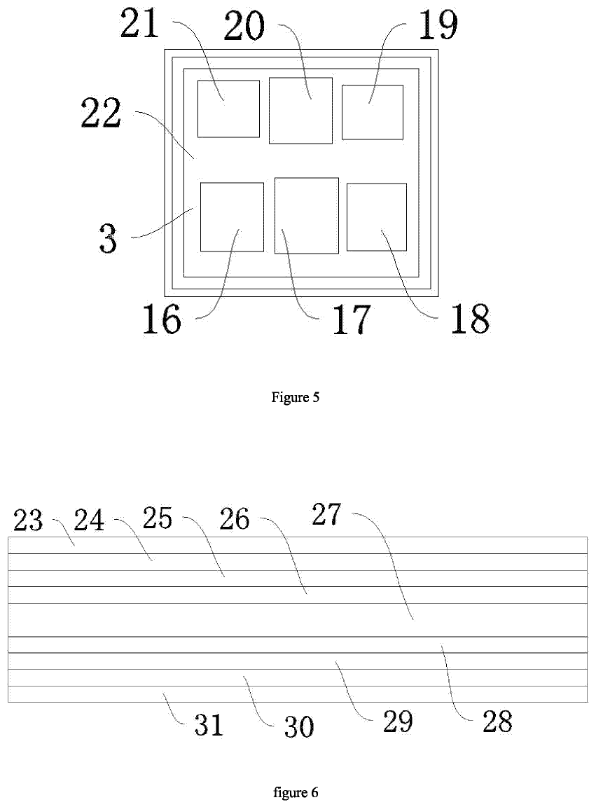

As shown in FIG. 1 to FIG. 7, a light-emitting device applicable to a notebook computer keyboard includes a power adapter 1, a control box 3, a keyboard membrane 6, a display screen 13, and a keyboard membrane 6 is provided with a button membrane 7, a keyboard The membrane 6 and the button membrane 7 are used to protect the keyboard, and the button membrane 7 and the keyboard membrane 6 are integrally formed. The button membrane 7 is provided with a display screen 13 for displaying and displaying the display screen 13 On the membrane 7, the button membrane 12 is provided with a button slot 12, and the button slot 12 and the button membrane 7 are integrally formed. The keyboard membrane 6 includes an upper antistatic layer 26, a central base layer 27 and a heat conduction layer 28, and the heat conduction layer 28 is used. To conduct heat, the upper antistatic layer 26 is disposed above the central base layer 27, the heat conductive layer 28 is disposed below the central base layer 27, the upper antistatic layer 26 is provided with an upper waterproof layer 25, and the upper waterproof layer 25 is provided with an antibacterial layer 24, which is antibacterial The layer 24 is used for antibacterial, and the anti-contamination layer 23 is disposed above the anti-bacterial layer 24, and the anti-contamination layer 23 is used for anti-contamination. The lower anti-static layer 29 is disposed under the heat-conducting layer 28, and the lower anti-static layer 29 and the upper anti-static layer 26 are used. To prevent static electricity, a lower waterproof layer 30 is disposed under the lower antistatic layer 29, and the lower waterproof layer 30 and the upper waterproof layer 25 are used for waterproofing and waterproofing. A nano-adhesive layer 31 is disposed under the 30, and the nano-adhesive layer 31 is used to facilitate the mounting of the fixed keyboard membrane 6, the anti-contamination layer 23, the anti-bacterial layer 24, the upper waterproof layer 25, the upper anti-static layer 26, the central base layer 27, the heat-conducting layer 28, The lower antistatic layer 29, the lower waterproof layer 30 and the nano-adhesive layer 31 are bonded by glue, and the other side of the keyboard membrane 6 is provided with a second connecting braided wire 8, and the second connecting braided wire 8 is embedded on the keyboard membrane 6. The other end of the second connecting braided wire 8 is provided with a control box 3, and the control box 3 is provided with a power switch 9, the power switch 9 is used for controlling the power supply, and the power switch 9 and the control box 3 are buckled, and the control box 3 is A circuit board 22 is provided, the circuit board 22 is fixed in the control box 3 by screws, the power switch 9 and the circuit board 22 are connected by wires, and the display screen 13 is connected to the circuit board 22 through the second connection braiding wire 8, and the control box 3 is connected. The other side is provided with a first connecting flat wire 4, the other end of the first connecting flat wire 4 is provided with a USB connector 5, the USB connector 5 is used for connecting the USB interface, and the USB connector 5 is provided with a connection indicator light 14 and a working status indicator 15, the connection indicator 14 and the working status indicator 15 are respectively embedded in the USB connector 5, the USB connector 5, the connection The indicator light 14 and the working status indicator light 15 are connected to the circuit board 22 through the first connecting flat wire 4, and the power supply extension cable 2 is disposed behind the control box 3, and the power adapter 1 is disposed at the other end of the power extension cable 2, and the power adapter 1 is used. To adapt the power supply, the power adapter 1 and the circuit board 22 are connected by a power extension cord 2, the power adapter 1 is provided with a power plug 10, the power plug 10 is used to connect the power source, and the power plug 10 is embedded in the power adapter 1, the power source A rubber spacer 11 is disposed between the extension cord 2 and the power adapter 1, and the rubber spacer 11 is used to protect the power extension cord 2, and the rubber spacer 11 is an interference fit with the power extension cord 2 and the power adapter 1 respectively.

A filter 20 is disposed on the circuit board 22, and the filter 20 is soldered to the circuit board 22 by soldering. The filter 20 is used to prevent signal interference, and the filter 20 and the circuit board 22 are electrically connected by soldering.

A Wi-Fi chip 21 is disposed on one side of the filter 20, and the Wi-Fi chip 21 is soldered on the circuit board 22 by soldering. The Wi-Fi chip 21 is used for receiving and transmitting wireless signals, and is provided between the Wi-Fi chip 21 and the circuit board 22 by soldering electrical connection.

The other side of the filter 20 is provided with a signal amplifier 19 which is soldered to the circuit board 22 by means of soldering, the signal amplifier 19 is used to amplify the enhancement signal, and the signal amplifier 19 and the circuit board 22 are electrically connected by soldering. Sexual connection.

A controller 16 is disposed in front of the Wi-Fi chip 21. The controller 16 is soldered to the circuit board 22 by soldering. The controller 16 is used for control, and the controller 16 and the circuit board 22 are electrically connected by soldering.

A processor 17 is provided in front of the filter 20 for processing signals and data; the processor 17 is soldered to the circuit board 22, both of which are electrically connected by soldering.

A memory 18 is provided in front of the signal amplifier 19 for storing data, the memory 18 is soldered to the circuit board 22 by soldering, both of which are electrically connected by soldering

The invention also provides a method for using a light-emitting device applicable to a notebook computer keyboard, which is applied to a light-emitting device applicable to a notebook computer keyboard, and the specific use method comprises the following steps:
a. the keyboard membrane 6 is pasted on the notebook computer keyboard through the nano-adhesive layer 31, the power adapter 1 is connected to the power source through the power plug 10, and the power switch 9 is turned on, and the USB connector 5 is connected with the USB interface of the notebook computer. The connection indicator light 14 is red, and the working status indicator light 15 is green after the installation is completed, and the connection indicator light 14 is turned off;
b. after being ready, the parameters and functions of the device can be set by using the notebook computer through the driver software, and the device related data can be stored in the memory 18, so that the parameters and functions set are still available after the notebook computer is replaced, and the repeated setting is avoided;
c. during the use process, the display screen 13 on the corresponding button membrane 7 can display the corresponding shortcut function icon according to the corresponding shortcut key according to the opening software, so that the software can be operated more conveniently, and the effect of getting twice the result with half the effort can be achieved at night. The display screen 13 can emit light and display its corresponding button name so that the notebook computer can be operated in a dark environment. When listening to music, the display screen 13 can emit colorful lights according to the rhythm of the music;
d. the Wi-Fi chip 21 can receive and transmit wireless network signals, the signal amplifier 19 can amplify the wireless signal, and the filter 20 can prevent signal interference. By setting, a wireless connection can be established between the mobile phone and the device, and a wireless network connection is established with the mobile phone. After that, the notebook can be turned on and off by the mobile phone and other corresponding operations, so that the notebook computer can be operated more conveniently, and the notebook experience is improved;
c. finally, the anti-contamination layer 23 and the anti-bacterial layer 24 can effectively resist the anti-staining and anti-bacterial, and the upper anti-static layer 26 and the lower anti-static layer 29 can prevent electrostatic damage to the electronic components, and the upper waterproof layer 25 and the lower waterproof layer 30 can ensure The keyboard is waterproof and the thermal layer 28 can conduct heat to assist the notebook computer in dissipating heat.

The basic principles, main features and advantages of the present invention have been shown and described above. It should be understood by those skilled in the art that the present invention is not limited by the foregoing embodiments, and that the present invention is only described in the foregoing description and the description of the present invention, without departing from the spirit and scope of the invention. Various changes and modifications are intended to be included within the scope of the invention as claimed.

1. A light-emitting device applicable to keyboard of notebook computer and use method thereof, comprising: a power adapter, a control box, a keyboard membrane, a display screen, wherein the keyboard membrane is provided with a button membrane, which is integrated processed on the button membrane amid, which is devised with the display screen, which is embedded on the button membrane with a button slot; the keyboard membrane comprises an upper antistatic layer, a central base layer and a heat conduction layer, the upper antistatic layer is disposed above the central base layer, and the heat conduction layer is disposed under the central base layer. An upper waterproof layer is disposed above the upper antistatic layer, an antibacterial layer is disposed above the upper waterproof layer, an anti-contamination layer is disposed above the antibacterial layer, and a lower antistatic layer is disposed under the heat conduction layer, the lower layer a lower waterproof layer is disposed under the antistatic layer, and a nano-adhesive layer is disposed under the lower waterproof layer, the anti-stain layer, the anti-bacterial layer, the upper waterproof layer, the upper anti-static layer, and the central base layer The heat conductive layer and the lower antistatic layer The lower waterproof layer and the nano-adhesive layer are bonded by glue, and the other side of the keyboard membrane is provided with a second connecting braided wire, and the second connecting braided wire is embedded on the keyboard membrane. The other end of the second connecting braided wire is provided with the control box, the control box is provided with a power switch, a snap connection between the power switch and the control box, and a circuit board is arranged in the control box The circuit board is fixed in the control box by screws, and the power switch and the circuit board are connected by wires, and the display screen is connected to the circuit board through the second connection braiding wire, The other side of the control box is provided with a first connecting flat wire, and the other end of the first connecting flat wire is provided with a USB connector, and the USB connector is provided with a connection indicator light and an operating status indicator, the connection indicator light and The working status indicator is respectively mounted on the USB connector, and the USB connector, the connection indicator and the working status indicator are connected to the circuit board through the first connection flat wire, and the control A power extension cord is disposed behind the box, and the electric The other end of the source extension line is provided with the power adapter, and the power adapter and the circuit board are connected by the power extension cable, and the power adapter is provided with a power plug, and the power plug is embedded in the power supply A rubber spacer is disposed between the power extension cord and the power adapter on the adapter, and the rubber spacer has an interference fit with the power extension cord and the power adapter, respectively.

2. A light-emitting device applicable to keyboard of notebook computer and use method thereof according to claim 1, wherein said circuit board is provided with a filter, said filter being soldered to said circuit board by soldering.

3. A light-emitting device applicable to keyboard of notebook computer and use method thereof according to claim 2, wherein a side of said filter is provided with a Wi-Fi chip, said Wi-Fi chip being soldered to said circuit board by soldering on.

4. A light-emitting device applicable to keyboard of notebook computer and use method thereof according to claim 3, wherein the other side of the filter is provided with a signal amplifier, and the signal amplifier is soldered to the circuit by soldering On the board.

5. A light-emitting device applicable to keyboard of notebook computer and use method thereof according to claim 4, wherein a controller is disposed m front of the Wi-Fi chip, and the controller is soldered on the circuit board by soldering.

6. A light-emitting device applicable to keyboard of notebook computer and use method thereof according to claim 5, wherein a processor is disposed in front of the filter.

7. A light-emitting device applicable to keyboard of notebook computer and use method thereof according to claim 6, wherein said processor is soldered to said circuit board by soldering.

8. A light-emitting device applicable to keyboard of notebook computer and use method thereof according to claim 7, wherein a memory is disposed in front of said signal amplifier.

9. A light-emitting device applicable to keyboard of notebook computer and use method thereof according to claim 8, wherein said memory is soldered to said circuit board by soldering.

10. A method of using a light-emitting device applicable to keyboard of notebook computer according to claim 1, the method including the following steps:
a. The keyboard membrane is pasted on the notebook computer keyboard through the nano-adhesive layer, the power adapter is connected to the power source through the power plug, and the power switch is turned on to connect the USB connector to the USB of the notebook computer. The interface is connected. After the connection is completed, the connection indicator is red. After the driver is installed, the working status indicator is green, and the connection indicator is off;
b. After the computer is ready, the parameters and functions of the device can be set by the driver software through the driver software. The device related data can be stored in the memory, so that the parameters and functions set are still available after the notebook computer is replaced, and the setting is avoided;
c. During use, the corresponding display screen on the button membrane can display corresponding shortcut function icons according to different shortcut keys according to different software, so that the software can be operated more conveniently, and the effect is more effective. At night, the display screen can illuminate and display its corresponding button name so that the notebook computer can be operated in a dark environment. When listening to music, the display screen can emit colorful lights according to the rhythm of the music;
d. The Wi-Fi chip can receive and transmit a wireless network signal, the signal amplifier can amplify the wireless signal, the filter can prevent signal interference, and the wireless connection between the mobile phone and the device can be established through setting, and the mobile phone is established with the mobile phone. After the wireless network is connected, the notebook computer can be turned on and off and other corresponding operations through the mobile phone, so that the notebook computer can be operated more conveniently, and the notebook user experience is improved;
e. Finally, the anti-contamination layer and the anti-bacterial layer can effectively resist anti-staining and anti-bacterial, and the upper anti-static layer and the lower anti-static layer can prevent electrostatic damage to electronic components, the upper waterproof layer and the The waterproof layer can ensure that the keyboard is waterproof, and the heat conduction layer can conduct heat, thereby assisting the notebook computer to dissipate heat.
